(19) United States
(12) Patent Application Publication
(10) Pub. No.: US 2010/0012124 A1
(43) Pub. Date: Jan. 21, 2010

(54) REBREATHER RESPIRATORY LOOP FAILURE DETECTOR

(76) Inventor: Alexander Roger Deas, Edinburgh (GB)

Correspondence Address: DARBY & DARBY P.C., P.O. BOX 770, Church Street Station, New York, NY, 10008-0770, US

(21) Appl. No.: 12/499,225

(22) Filed: Jul. 8, 2009

Related U.S. Application Data
(60) Provisional application No. 61/129,605, filed on Jul. 8, 2008.

(51) Int. Cl. A61M 16/20; A61B 5/097; G08B 17/10; G08B 21/00

(52) U.S. Cl. 128/204.22; 600/532; 340/632; 340/626

(57) ABSTRACT

A rebreather safety monitoring device comprising a carbon dioxide sensor provided with a gas sampler adapted for sampling a gas in a rebreather breathing loop from a location between an inhale one-way valve on a rebreather mouthpiece and a carbon dioxide scrubber and providing the obtained gas sample to the carbon dioxide sensor, and a means to provide alarms or warnings based on the level of the expired carbon dioxide. The present invention detects a wide range of failures of a rebreather by measurement of the expired carbon dioxide level and application of that level to trigger alarms, provide loop shut-off or provide safety warnings.

### CROSS REFERENCE TO RELATED APPLICATIONS

The present application claims the benefit of U.S. Provisional Patent Application Ser. No. 61/129,605, filed on Jul. 8, 2008, which is hereby incorporated by reference in its entirety.

### BACKGROUND

Rebreathing equipment is used for human life support in adverse environments, such as in diving underwater, hazardous materials handling and for manned activities in space outside of a spacecraft.

Rebreathers operate by circulating the user's expired gas through a loop comprising counterlungs, a carbon dioxide scrubber and a means to inject oxygen to make up for that lost through metabolism or vented from the loop, then back to the user to inspire.

The critical nature of the scrubber unit in the rebreather has given rise to multiple proposals for a Carbon dioxide (CO2) sensor to monitor inspired gas. For example in the European Standard for rebreathers EN14143:2003, a detailed performance requirement is stated, and the means by which that is tested, for inspired gas carbon dioxide sensors. OSHA regulations contain reference to a similar such device. There is also a problem with inspired CO2 sensors in that no company has managed to create an inspired CO2 sensor that works in practice to date

The limitation of these inspired CO2 sensors, if they were to be reduced to practice, is that they are limited to monitoring inhaled gas to detect scrubber failure or scrubber breakthrough. Unfortunately these two failure modes are just two out of nine failure modes in a rebreather that involve CO2. The full list includes:
1. Scrubber breakthrough
2. Scrubber bypass
3. Flooding of the loop causing an increase in breathing resistance
4. Flooding of the loop causing scrubber failure
5. Foreign material blocking any part of the breathing loop
6. One way valve failure (flapper valve failure)
7. Excessive Work Of Breathing
8. Excessive dead volume
9. The user has a physiology that causes them to retain CO2 more than normal.

Serious or fatal accidents appear to have occurred due to each of these root causes, in some cases a series of accidents. The population of active rebreather users does not appear to exceed 10,000 in number, so it would appear these failure modes occur frequently. Many rebreather users also report having experienced one or more of the above failure modes but survived: this reinforces the view that these failure modes occur frequently.

It is possible to add additional sensors to a breathing loop to detect specific failure modes, but it is generally uneconomic. A good example is the one way valves, which are typically mushroom valves. These valves can tear, fail to be installed, catch on the web that holds the valve, or stick either open or shut in the presence of foreign matter. A simple method of detecting mushroom valve failure is to place an infra red LED inside the mouthpiece and sensors on either side of the mouthpiece—in the breathing hoses. The output of the sensors should show a peak not less than once every ten seconds, and should not both show a signal at the same time. The problem with this method of monitoring is that it is very specific to the mouthpiece and does not indicate any failures due to excess dead volume or work of breathing. It is a large cost for just one failure mode. It is also liable to malfunction due to water ingress or detritus on the LED or sensor.

What may appear to be fixed features, such as the dead volume when the user is breathing from a mouthpiece, may not be fixed in all applications; for instance, when an oro-nasal mask is used the dead volume depends on how well the mask fits the user, and how hard the user presses their face into the mask. That is, the range of causes for a high retained CO2 is broader than simple equipment design issues.

These CO2 related failure modes quickly disable the user due to an inherent positive feedback loop. Increases in retained CO2 cause the user to breathe faster. This causes an increase in the Work Of Breathing (WOB) of the breathing loop. If the WOB increases, the users retained CO2 increases, causing their respiratory rate to increase, causing the WOB to increase further. This positive feedback loop is limited by either the user collapsing or the user switching to a task which requires much lower energy. The positive feedback nature of CO2 retention causes these fault modes to be particularly pernicious in their onset and progress, disabling the user by the time they become aware of what the problem is.

It is not practical to measure blood carbon dioxide levels directly, using light transmission or backscatter sensors for example, because there are often large changes in the circulation of the user: rebreathers are often used in cold environments, or underwater. It is also undesirable to pinch the user if the user has to complete a decompression profile, as the pinched area would be more liable to decompression damage if the pressure were to vary during the dive.

The problems of monitoring anything other than inhaled CO2 in a rebreather are compounded by the technical difficulties of measuring CO2 in practice in a rebreather. There have been many attempts to use CO2 monitors for rebreathers over the years. The only companies believed to have implemented this successfully are the British company HSM Engineering Ltd for restricted applications of monitoring inhaled gas for nitrox and pure oxygen rebreathers, and another British company, Deep Life Ltd, for commercial diving rebreathers using methods that have been maintained hitherto as a trade secret, and are revealed herein.

Carbon dioxide sensors to detect breathing and measure respiratory parameters have been in use for decades, such as in multi-patient systems that were in hospital service during the 1990s. These have fallen out of fashion in a surgical setting, but several systems are still available, such as the Innocor from Innovision AS, Denmark: one Innocor Respiratory Monitor provides a full respiratory analysis from inspired and expired gases, on a breath by breath basis, using infra-red absorption to measure carbon dioxide in a clinical environment. The same company produces a similar product using mass spectrometry. The function of these systems is to measure the respiratory function of a patient in a clinical environment: that is to measure the tidal volume, respiratory quotient, respiratory rate, anabolic threshold etc. Some inventions such as that in US2002/0104536 seem to use a subset of these functions as a respiration detector: the method in widespread use in a diving setting is to detect the fall in the partial pressure of oxygen (PPO2).

The most common method of detecting CO2 is by infra-red absorption: most gases can be detected by this means, just by placing an optical filter of the wavelength at which the gas absorbs infra-red in front of an infra-red sensor, with the gas to be sampled in a path between the sensor and an infra-red emitter having enough energy at the frequency at which absorption occurs. The infra-red absorption spectra for CO2 has been well known, with sensors generally using absorption peaks around the 4.260 nm wavelength (2349 cm⁻¹). The spectra across a wide spectral range has been published by V. F. Golovko, “Calculations of carbon dioxide absorption spectra in wide spectral regions,” Atmospheric and Oceanic Optics, 14, pp. 807-812, 2001.

Infra-red absorption CO2 sensors have been commercially available for decades. Single wavelength systems such as that described in CA2068081 were common until the 1980s, when dual channel sensors were introduced. The dual channel sensor compares the amplitude of the received infra-red channel through a filter that passes the absorption band of interest, with that of another channel with an absorption band where the gas of interest does not absorb significant amounts of infra-red energy. The British company Analox Ltd even produces a range of dual channel sensors suitable for hyperbaric use. However, serious problems remain for carbon dioxide sensors to operate reliably in a rebreather, including:
1. Pressure causes a spectral broadening of the infra red absorption bands normally used to detect carbon dioxide. The broadening typically reduces the magnitude of the received signal by 300% over the range of depths used for manned underwater operations. The pressure broadening effect on CO2 spectra is well known.
2. Inert gases, especially helium, cause a narrowing of the spectrum, which can result in a large change in the sensitivity of absorption infra-red sensors. This effect is well known, and described in papers such as that by Golovko, “Modeling of IR absorption spectra of the mixture CO2-He at moderate and high pressures”, Tenth Joint Int. Symp. on Atmospheric and Ocean Optics/Atmospheric Physics I: Radiation Propagation in the Atmosphere and Ocean, G. G. Matvienko, G. M. Krekov, SPIE Vol. 5396SPIE, Bellingham, Wash., 2004)·0277-786X/04/$15·doi: 10.1117/12.548204.
3. Humidity in a rebreather is normally over 70%, and condenses. This is particularly the case for sensors for inspired gas, where the sensor is downstream of the scrubber. The reaction in a carbon dioxide scrubber generates water vapour.
4. Inert gases cause the infra-red emitter to cool, which modifies the spectrum: this can change the response by orders of magnitude. The correction to the received signal used in WO 02/036294 by the present inventor can be insufficient, with some emitter types, to produce a useable output with the required sensitivity at deep depths, when the infra-red emitter is sealed to prevent helium ingress but helium migration into the compartment around the emitter can be problematic.
5. The gas leaving the scrubber is warm: typically 35C to 95C in temperature, and the entrained water vapour in particular gives off Planck radiation that can be in the detection band. This creates a noise floor that can interfere significantly with the detection of carbon dioxide.

Basic carbon dioxide sensors such as GB2394281 simply do not work in practice due to these problems. An earlier invention by the present inventor, WO 02/036294, addressed the problems by correcting the received signal for pressure and helium, using hydrophobic membrane and the scrubber heat to keep the sensor dry. Others, such as Rose in US2007/0090290, dry the gas by using an injected gas or pressure expansion to create a dry gas that avoids condensation on the sensor.

Other methods such as fluorescent, sol-gel and chemical change sensors have been proposed on some internet forums for measuring inhaled rebreather gas, but none have been brought to a state where they have worked.

Methods involving heated sensors tend not to work in a rebreather due to the condensing humidity in the loop and the large differences in heat loss caused by the use of a variable fraction of helium as the make-up gas in the loop.

### OBJECT OF THE PRESENT INVENTION

It is an object of the present invention to detect a wide range of failures that cause carbon dioxide retention in rebreathers.

It is a further object of the present invention to detect one-way valve failure in rebreathers.

It is a further object of the present invention to detect when there is a failure of other parts of the breathing loop causing an increase in retained carbon dioxide levels due to excessive work of breathing.

If is a further object of the present invention to detect failures that cause an increase in the dead volume that affects the safe operation of a rebreather.

It is a further object of the present invention to detect carbon dioxide in the presence of helium, and under pressure, in a humid environment.

### SUMMARY OF THE PRESENT INVENTION

In one aspect of the invention, a rebreather safety monitoring device is provided, comprising:
a carbon dioxide sensor provided with a gas sampler, wherein the gas sampler is adapted for sampling a gas in a rebreather breathing loop from a location between an inhale one-way valve on a rebreather mouthpiece and a carbon dioxide scrubber and providing the obtained gas sample to the carbon dioxide sensor, and
a means to provide alarms or warnings based on the level of the expired carbon dioxide.

In one embodiment of the present invention, the carbon dioxide sensor is calibrated using the carbon dioxide level in human expired gas.

In one embodiment, the carbon dioxide sensor is fitted with a hydrophobic membrane

In one embodiment of the present invention, the carbon dioxide sensor is a dual channel infra-red absorption sensor.

In one embodiment, the carbon dioxide sensor is powered up with a low periodic duty cycle, but where the alarm system is powered even when the carbon dioxide sensor is powered down.

In one embodiment of the present invention, the carbon dioxide reading is powered up and a reading taken when a change in the user's respiratory rate is detected. Preferably, that is augmented by a pressure sensor that is applied to compensate the carbon dioxide reading for changes in ambient pressure. Alternatively, that is augmented by a helium sensor that is applied to compensate the carbon dioxide reading for changes in the partial pressure of helium.

In one embodiment of the present invention, the carbon dioxide sensor is a dual channel infra-red absorption sensor with means to drive the infra-red light source with greater power when under ambient pressure or in the presence of gases having a high thermal capacity, than in air at one atmosphere pressure, thus stabilising the spectrum of the emitted light under a range of pressures or in the presence of helium.

In another embodiment of the present invention, the carbon dioxide sensor is a dual channel infra-red absorption sensor with the infra-red emitter isolated from the effects of pressure and helium such that the emitted spectrum does not change by more than 50% under the range of operating pressures or helium the monitor covers.

In one embodiment of the present invention, the alarm level is used to drive shut a valve that closes the breathing loop.

In one embodiment of the present invention, the alarm level is used to drive shut a valve that closes the breathing loop at the mouthpiece and switches the user to an alternative gas source.

In one embodiment of the present invention, the absence of carbon dioxide, or the presence at a partial pressure lower than that normally expired by a human, is applied to trigger a warning or alarm level.

In one embodiment of the present invention, the presence of carbon dioxide at a partial pressure higher than that normally expired by a human is applied to trigger a warning or alarm level.

In one embodiment of the present invention, the circuitry is integrated with a partial pressure of oxygen monitor or measurement device or controller.

In one embodiment of the present invention, the carbon dioxide sensor is coupled thermally to the scrubber such that it operates at more than 3 degrees Celsius above the ambient temperature.

In one embodiment of the present invention, the monitoring device is calibrated automatically when the scrubber canister is opened and the scrubber removed, as indicated by the presence of light on a light sensor, with a human interface that allows the background level of carbon dioxide to be indicated to the calibration system.

The present invention thus provides for a carbon dioxide sensor using the level of expired CO2 as a means to detect the presence of a broad range of rebreather safety hazards, including failure of rebreather one-way valves, excessive work of breathing, scrubber failure, and excessive dead volume. These failures are implied by the present invention from the rise in the partial pressure of carbon dioxide of the user's exhaled gas, by measurement of the partial pressure of carbon dioxide and application of the amplitude of that signal to trigger alarms or provide warnings.

### BRIEF DESCRIPTION OF THE INVENTION AND FIGURES

The invention will now be described by way of example, without limitation to the generality of the invention, and with reference to the following figures:

FIG. 1 shows an example of a rebreather loop with a CO2 sensor fitted according to the present invention, comprising a user (1), who breathes into a mouthpiece or oro-nasal mask (2) containing two one-way valves that regulate the flow direction around the breathing loop such that exhaled gas is channelled to an exhale counterlung (4), and then to a scrubber (6). A CO2 sensor (5) suitable for rebreather applications is located between the exhale port of the mouthpiece (2) and the scrubber (6). The rebreather loop contains an injector valve (7) for dosing the loop with an oxygen bearing gas from a gas cylinder (8), it may contain a further inhale counterlung (9), a loop over-pressure valve (3) shown here on the inhale counterlung but many rebreathers may locate the over-pressure valve (3) on the exhale counterlung, a means to add a make-up gas to the loop, shown here using an Automatic Diluent Valve (ADV) (11) from a second cylinder (10), and a breathing path to the inhale one-way valve in the mouthpiece (2).

FIG. 2 shows a block diagram of a typical infra-red CO2 sensor circuit comprising a signal generator or power source (12), driving an Infra-Red light source (13) such as a silicon micromachined IR source or a lamp, which produces an infra-red output at a wavelength that is absorbed by CO2 and at another wavelength that is not absorbed so significantly. The emitted Infra-Red signal is allowed to pass through sufficient length of path containing the gas (14) to be measured (typically 30 to 60 mm), and the infra-red signal that has passed through the gas to be measured is detected by an infra-red sensor or detector (15) containing filters, prism or grating to produce two signals, one of which is proportional to the amplitude of the signal at the wavelength absorbed by CO2 and the other at the reference wavelength which is absorbed less strongly or not absorbed, which are then amplified by amplifiers (16) and (17), applied to a divider (18), the output of which is a ratio of the energy in the two signals: CO2 absorption channel and reference channel. A means to correct the sensor output value is applied (19) using data from the environment such as depth or gas type, and the output is a signal proportional to the partial pressure of CO2 in the infra-red light path. The amplifiers are normally a low drift chopper equivalent type, and may be AC coupled where the signal source (12) can be commutated quickly.

FIG. 3 shows the waveforms in one example embodiment of a rebreather carbon dioxide sensor according to FIG. 2, where the absorption of infra-red energy is used to detect the level of carbon dioxide. In FIG. 3a when the Infra-Red source is switched on, the output of the divider (18) shows a large negative drop, which peaks 360 ms after the light source is switched on, due to thermal dynamics of the infra-red light source (13), gas thermodynamics and re-emission effects within the gas. The negative peak of this signal is proportional to the CO2 gas concentration. In FIG. 3b, when the infra-red light source is switched off, there is an instant extinction of the reference path due to no energy being absorbed by the gas at that wavelength but the CO2 re-emits energy causing a positive peak in the output signal. The positive peak can be used as an alternative to the first peak on switch on, or to obtain greater accuracy out of the sensor system by averaging it with the first peak, reducing noise.

FIGS. 3c and 3d show the actual reference signal and actual CO2 signal from the reference signal amplifier (16) and CO2 signal amplifier (17) respectively, as a function of time for a series of four short light pulses. Note that the sensor amplifiers can be AC coupled, as in this case, which reduces problems of drift.

FIGS. 4a and 4b show two different views of a moulded plastic shell around which a hydrophobic membrane can be welded, to protect the carbon dioxide sensor from humidity. The shell sits on a circuit board using an O-ring seal, and the other end is in contact with the scrubber canister in this example embodiment. The dimensions of the shell are in this case 75 mm in diameter: other dimensions can be scaled.

FIG. 5 shows a block diagram of a circuit to overcome the effect of pressure and helium on the sensor, by overdriving the infra red emitter to maintain a constant amplitude of the decay signal in the reference channel. This is the same as for FIG. 2, except there is an additional signal (21) that keeps the reference channel at a constant amplitude. A closely related embodiment uses a feedback signal taken from the correction unit (19) where the feedback signal can be checked to ensure it will result in the IR source operating within a safe operating envelope by using an additional pressure sensor (internal to the correction unit 19), and helium sensor (internal to the correction unit 19), such that the infra-red light source (13)—this will allow the IR source (13) to be driven with significantly more power than would be safe at one atmosphere pressure in air when under pressure or in the presence of a sufficient pressure of helium, as these media tend to cool the sensor such that considerably more energy may be required to maintain the infra-red light output at the wavelengths of interest (reference channel and CO2 channel). It is advantageous but not essential to have the reference channel at a longer wavelength that the CO2 channel, as when the Infra-Red sensor cools, the longer wavelengths seem to diminish in amplitude first—this is contrary to what would be expected but is the observation with several types of infra-red emitter in laboratory experiments.

FIG. 6 is a UML description of the calibration and measurement algorithm for an example embodiment of a carbon dioxide sensor, the block diagram for which is described by FIG. 5.

### OPERATION OF THE PRESENT INVENTION

The operation of the invention will be described, by reference to example embodiments without limit to the generality of the invention.

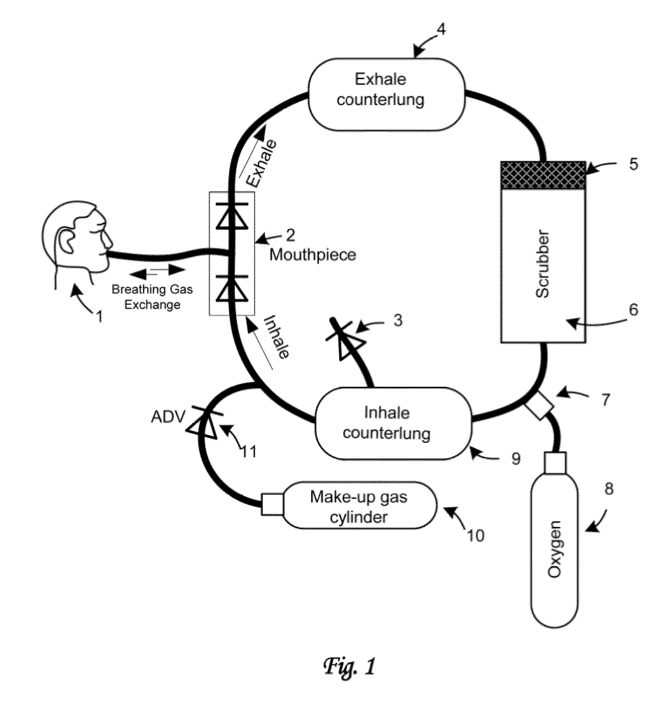

The functionality of the present invention should be apparent to a person skilled in the art of rebreathers and sensor electronics from FIGS. 1 to 5, the brief description above, in conjunction with the following description of the operation of the present invention.

For brevity, the examples will assume the user is a diver, and the rebreather is the closed loop type rather than semi-closed in that it uses both a make-up gas and an oxygen containing gas to maintain the loop PPO2, though the invention can be applied to the widest range of rebreathers including pure oxygen rebreathers and to semi-closed rebreathers without material modification. A loop type rebreather will be described, though by the application of a masking gate function to the sensor signal, or by use of averaging, the invention can be applied to pendulum type rebreathers. The example rebreather will use two counterlungs, but the invention is equally applicable to single counterlung rebreathers.

The partial pressure of CO2 (PPCO2) in exhaled gas is normally around 0.04 ATM, but under heavy work the respiratory quotient increases, and the exhaled gas can contain 0.06 ATM of CO2 without there being any fault condition in the rebreather. Where there is no scrubber fitted, the second or third breath will see the exhaled PPCO2 increase to 0.08 ATM or more.

The failure of the breathing loop can be indicated to the user using a tricolour LED. For example, using a Blue/Red/Green LED the CO2 information status may be communicated as:
If 0.005 ATM<PPCO2 then LED off. This indicates there is no breathing detected in the loop, or there is a complete failure of the inhale one way valve.
If 0.005 ATM<PPCO2<0.035 ATM, the Green led is flashing, with 500 ms flashes, 250 ms pauses, then repeat on an 8 second cycle. Each flash means the PPCO2 is 0.005 and, so 0.03 would be 6 green flashes; Values are rounded to the nearest 0.005. This means that the loop should be checked, in particular for partial or complete failure of the inhale valve.
If 0.035 ATM<PPCO2<=0.060 ATM, the blue led is flashing with 500 ms flashes, 250 ms pauses, then repeat on an 8 second cycle. Each flash means the PPCO2 is 0.01 so 0.06 would be 6 flashes; Values are rounded to the nearest 0.01. Values in this range are normal.
If 0.060 ATM<=ppCO2<0.065 ATM, there is blue solid light; This is the maximum safe level.
If ppCO2>=0.065 ATM, there is red solid light and the rebreather should preferably close the breathing loop to prevent the user breathing from it while in this hazardous state, such as by an automatic loop shut off valve described by the invention U.S. Pat. No. 6,817,359 with the shut off valve located preferably after the inhale one-way valve in the mouthpiece (2).

Where the CO2 sensor has a high power consumption, it is of benefit to combined the CO2 monitoring with a respiratory monitor, such as that described in GB0516751.5 to reduce the power consumption and provide a very rapid response to a change in breathing pattern: any increase in retained CO2 causes an automatic change in breathing rate.

The fault modes are detected by the expired CO2 monitor as follows:
Scrubber breakthrough causes a rise in the inspired CO2, and a similar rise in expired CO2. The body acts as an averaging mechanism, that reduces false alarm rates that are present if a pure level based inspired CO2 sensor is used: scrubbers often breakdown momentarily well before they become exhausted, due to surface area saturation effects in the scrubber. For example, if a person working very hard is producing a PPCO2 of 0.065 ATM, a scrubber breakthrough would increase this above alarm thresholds. A user who is working more gently may tolerate a higher degree of breakthrough, and an inspired PPCO2 of 0.03 ATM is not unreasonable as a safety limit in that case.
Scrubber bypass causes a similar rise in inspired and expired CO2 as for scrubber breakthrough above.
Flooding of the loop causing an increase in breathing resistance, which is only detectable either directly as water in the loop (which would require multiple sensors) or as an increase in expired CO2. The expired PPCO2 will exceed 0.065 very rapidly in the event of a serious loop flood.
Flooding of the loop causing scrubber failure. This is detected in the same manner as loop flooding, and later, as scrubber breakthrough above.
Foreign material blocking any part of the breathing loop. This results in an increased Work of Breathing, causing increased retained CO2, and an increase in expired CO2. The PPCO2 will increase to above 0.065 ATM rapidly if there is a significant loop blockage.
One-way valve failure (flapper valve failure). Where the inhale or exhale one-way valves fail in the mouthpiece (2), the PPCO2 will fall to an unusually low level. This should indicate a problem to the user, and can be checked for automatically during pre-dive checks: the rebreather controller or monitor can check that the expired PPCO2 is above 0.03 ATM. The location of the CO2 sensor has a material bearing on the ability to detect this failure mode reliably: if the sensor is in the exhale counterlung or too close to the mouthpiece, it will generally not be detected in every case. However, if the CO2 sensor is located between the exhale counterlung and the scrubber as shown in FIG. 1, then this mode can be detected very reliably.
Excessive Work Of Breathing, causes an increase in retained CO2 at depth, or when the user is working hard. This shows as an increase in expired CO2 levels and can be detected easily.
Excessive dead volume, causes an increase in the expired CO2 that travels to the exhale counterlung, and hence to the expired CO2 sensor. This high reading will vary depending on work levels, but it will be noticeably higher and will trigger the suggested 0.065 ATM alarm level before it is a disabling factor.
The user has a physiology that causes them to retain CO2 more than normal. The user will have a high expired PPCO2 level when performing work at depth. This will require the user to slow down and take more caution than average diver.

The above failure modes may occur singularly or multiple modes may occur at the same time. The important parameter to track is the blood CO2 level, which is well represented in the expired PPCO2 level which is measured by the present invention and used as the basis of an alarm system.

A problem that plagues CO2 sensors for measuring inhaled CO2 is that of calibration. Even the background ambient carbon dioxide level, currently averaging around 380 ppm, undergoes significant seasonal variations. In addition to that are changes in carbon dioxide levels due to nearby machines, flames, poor building ventilation and other factors. The result is that background CO2 levels vary from 200 ppm to 800 ppm, depending on location and the environment. This is a ratio of 4:1, so any extrapolation from that calibration point would produce an error of up to 400%. These large tolerances in the calibration gas makes inhale side CO2 sensors almost unworkable in an operational context, without regular recourse to laboratory calibration using trace gases.

It is possible to calibrate an inspired CO2 sensor if the user is able to provide a reference ambient carbon dioxide reading taken independently that is entered by a menu on a micro-controller display integrated with the device. However, this can cover only those environments where either an independent calibrated CO2 monitor is used or CO2 ambient data is available.

This calibration problem can be overcome in the present invention by calibrating the CO2 sensor to the partial pressure of CO2 in the exhaled gas from a relaxed user, which is normally between 0.035 and 0.04 ATM: an error band of just 14%. By the user breathing out into the exhale one-way valve in the mouthpiece (2) with the inhale side of the rebreather disconnected, a known gas can be applied to the CO2 sensor for calibration purposes, which has a very much smaller tolerance than the gas in the ambient environment, as a fraction of the full scale or range of the sensor. This process can be initiated automatically in the present invention by detecting the scrubber is open and the scrubber is removed, such as by detecting the presence of light: the inside of a scrubber canister is normally darker than the ambient light level. The user can then be prompted to exhale into the sensor assembly to obtain a reading of a users rest expired CO2 level as a calibration gas.

Attention will now turn to the practical issue of how to measure CO2 in a rebreather.

The level of carbon dioxide may be measured by some new forms of sol-gel sensor directly, but these are not readily available at the present time. There are micro-miniature phase fluorometers available commercially, but these appear to suffer significant problems in a rebreather type environment at the present state of this technology, particularly aging and contamination of the sol-gel. However, when those technologies mature, a similar method as described here can be used with those sensors, including protection from humidity, power management and multi-variate analysis to improve the signal to noise ratio under fluctuations of temperature and pressure.

The method for measuring CO2 that the invention uses in an example embodiment relies on the absorption of infra-red energy by carbon dioxide.

There are many possible circuits for a carbon dioxide sensor involving the measurement of the ratio of the signal strength between a measurement channel and a reference infra-red channel, where the measurement channel has a filter at an absorption peak for the desired gas, namely for carbon dioxide, and the reference channel has a nearby frequency that is absorbed to a much lesser extent by carbon dioxide. Sensors fitted with these filter combinations are readily available commercially.

The unique challenge for a sensor operating in a rebreather is to overcome the thermal noise, and variable effects of pressure and inert gases. The following description will therefore focus on an example embodiment using a circuit that includes pressure and helium compensation functions.

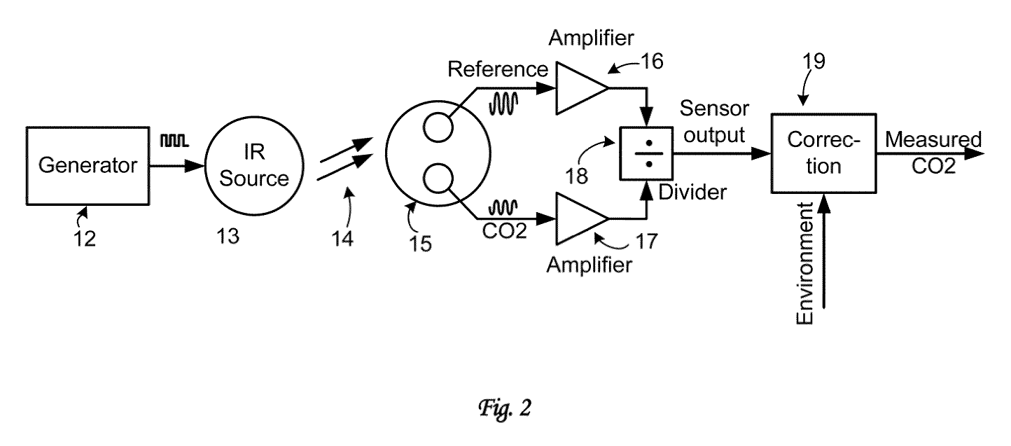

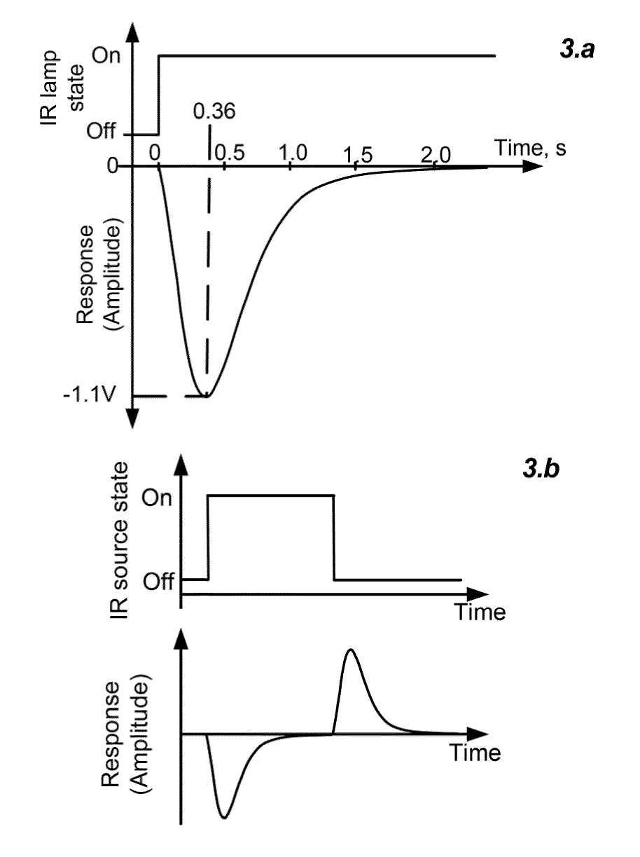

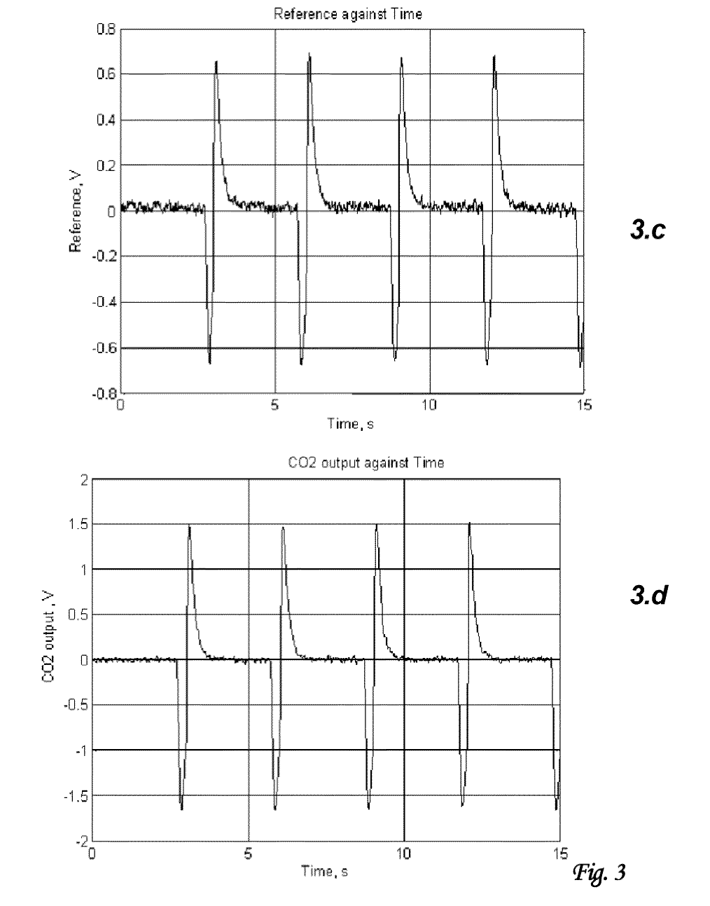

An example embodiment of a suitable carbon dioxide sensor is shown in FIG. 2, along with its related operating waveforms, mechanics and algorithm description shown in FIGS. 3 to 6 inclusive. The circuit implementing that block diagram and its operation would be apparent to a person skilled in the art of rebreathers and sensor electronics from those figures, but there are some particular features that are important, to which the reader's attention will now be drawn.

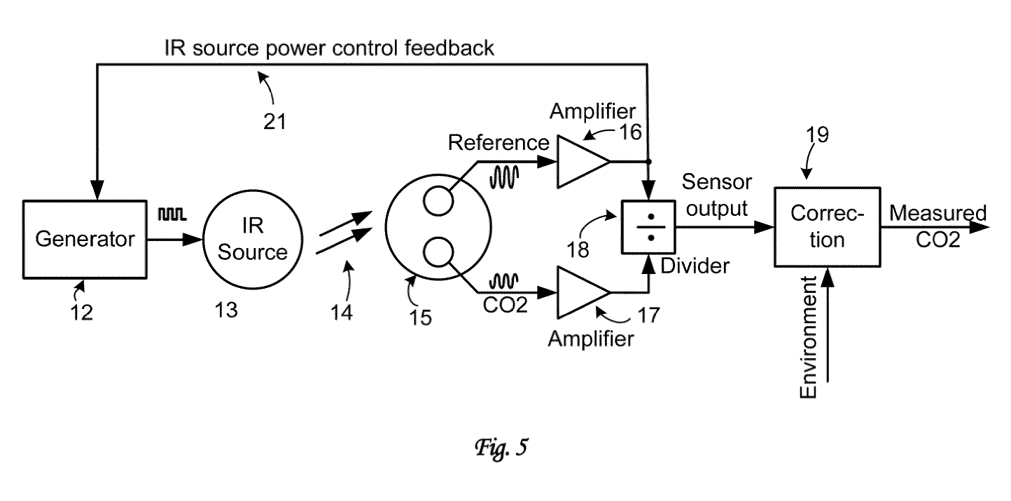

In FIGS. 2 and 5, the CO2 sensor drives an infrared source (13) from a signal generator (12), across a gas path (14), to detect the received signal using a detector (15) with amplifiers (16) and (17), divider (18), and correction block (19). The light path (14) should be protected from ambient light and condensation, such as by using a black tube for the path, surrounded by an external hydrophobic membrane. It is important there is a sufficient length of the light path (14) to contain sufficient CO2 molecules to produce a marked absorption response. In CO2 sensors designed for use in office type environments the light path often uses mirrors to cause the light to reflect backwards and forwards to provide a longer light path than the dimensions of the physical measurement chamber. In a rebreather the path should ideally be straight, as the effects of and condensation on the mirror will be very pronounced. Path lengths of 35 mm have been found to be sufficient, given careful low noise design of the circuit stages.

The divider (18) may be realised digitally by sampling the outputs of amplifiers (16) and (17), and applying an analogue to digital conversion process. Alternatively, it is possible to implement the ratio circuit using analogue differential amplifiers.

A thermal connection of the sensor light path to the scrubber has been found to be of benefit in raising the temperature of the gas path, to further exclude moisture and reduce the risk of condensation.

The optical filters in the detector (15) pass a wavelength of light that is inside the carbon dioxide absorption band. Sensors are available commercially with filters centred at 4.260 nm (2349 cm⁻¹) for the detector CO2 channel, with a second wavelength of IR light that is outside the CO2 absorption band for use as the reference channel of the detector (15) at 3.900 nm or the carbon monoxide band at 4.700 nm. Other CO2 spectral bands include 15 um (667 cm⁻¹), but producing a stable IR source at that wavelength in portable equipment is difficult. The reference channel should preferably avoid the water absorption spectra with wave numbers from 1000 to 2000, and avoid the absorption spectra for other gases that may be present.

The correction unit (19) adjusts the divider output to compensate for variations in the environment parameters that affect the divider output. The list of the compensated parameters can include ambient temperature, thermal shifts due to gas law effects, pressure, humidity, type of the gas, and circuit parameters. In some cases, it can be advantageous to generate the IR source power control feedback signal (21) from the correction unit, where limits can be introduced to provide an extra degree of safety to avoid applying too much power to the IR source (13).

The examples of the detector responses are shown in FIGS. 3.a to 3.d. A step input applied to the IR source generates one pulse shown in FIG. 3.a in each detector output channel. Each pulse of the IR source generates two pulses per detector channel shown in FIG. 3.b. The response of the IR source pulse sequence measured in the reference and the CO2 channel of the detector are shown in FIGS. 3.c and 3.d. The amplifiers (16) and (17) are normally chopper type DC amplifiers, but may include a high pass filter to remove the effects of DC offset and drift, as was used in the circuit that generated these waveforms.

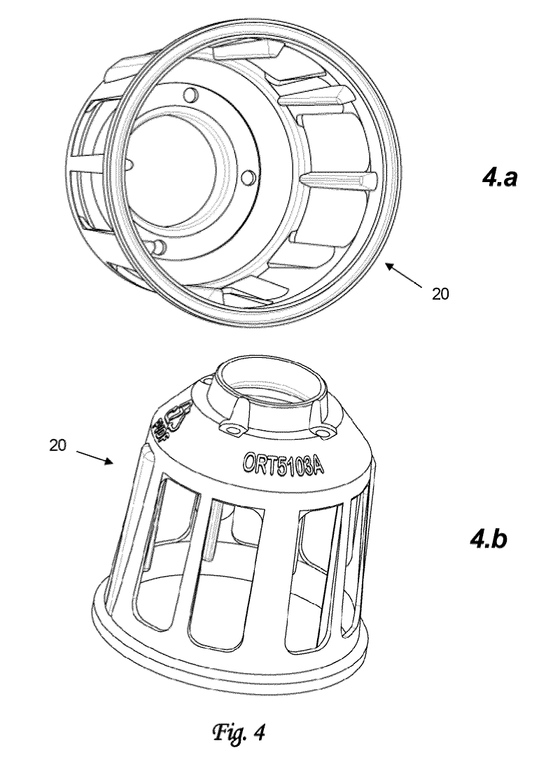

It is critically important to keep liquid water from condensation or from cleaning processes out of the light path (14) between the IR source (13) and the detector (15). Liquid water strongly absorbs IR radiation at the same wavelengths as CO2, so even very small water droplets anywhere in the sensing cell will generally cause erroneously high gas concentration readings. To protect the light path and electronics against water, the shell shown in FIG. 4 is fitted with a gas permeable hydrophobic membrane, such as Zitex A105 or GE PTFE based hydrophobic membrane. These membranes can be welded to suitable plastics from which the shell can be moulded, such as PVDF (Kynar), and low off-gassing of Polypropylene: PP that is free of plastisizing and softening agents, to form a chamber that prevents water ingress to the CO2 sensing area, but allows CO2 to pass freely.

The light power of the IR source depends on the thermal conductivity of the ambient gas. In FIG. 5, the feedback from the reference signal amplified by the amplifier (16) is used to control the source generator (12) to increase the amplitude of the source power supply when the reference signal is less than the set level and decrease the power supply when the signal is more than the set level.

The IR source (13) can be of any of several different types, including infra-red bulbs and silicon micro-machined infra-red sources. If helium or high pressure gas is in contact with the infra-red source, it will cool relative to the temperature the source operates at in normal atmospheric conditions (standard pressure, temperature, dry). This cooling will change the spectrum of the emitted infra-red light: in most cases, the longer wavelengths will be attenuated. If there is no energy being emitted in the absorption band, then the entire sensor will not work. It is important therefore to maintain the spectrum of the IR source (13) constant, or reasonably constant. There are two methods by which this can be achieved:
1. Protection of the IR source (13) from pressures and inert gases, such as by sealing the IR source in a chamber maintained at one atmosphere with a sapphire window through which light passes down the measurement path (14). This solution is expensive and prone to fail, particularly through helium ingress.
2. Increasing the power to the IR source (13) to maintain the spectrum of the emitted light. This can be done effectively, but generally requires enough power that if it were applied to the IR source (13) at one atmosphere in air, the operating life of the emitter would be reduced very drastically. For example, one particular silicon micro-machined emitter that is commercially available and provides a strong IR source across the spectrum covering 3.900 nm to 4.700 nm wavelengths, has a power supply of 3V0 at one atmosphere in air, with an absolute maximum supply rating of 3V3. Application of more than 3V3 at one atmosphere pressure in air will destroy the device, almost instantly. In 60 bar of helium, a power supply voltage of 7V3 has to be applied to obtain the same spectrum: the device is not damaged by this, because its operating temperature is the same as with 3V0 in air. The feedback channel (21) allows the power to the IR source (13) to be increased in this manner. To avoid sudden failure of the emitter due to a water droplet or dust attenuating the reference channel, it is advantageous in some embodiments to generate the source power feedback signal (21) from the correction unit, where the partial pressure of helium (PPHe) and ambient pressure can be measured such that the feedback signal is always within a safe operating envelope; this is an alternative feedback path to that shown in the embodiment in FIG. 5

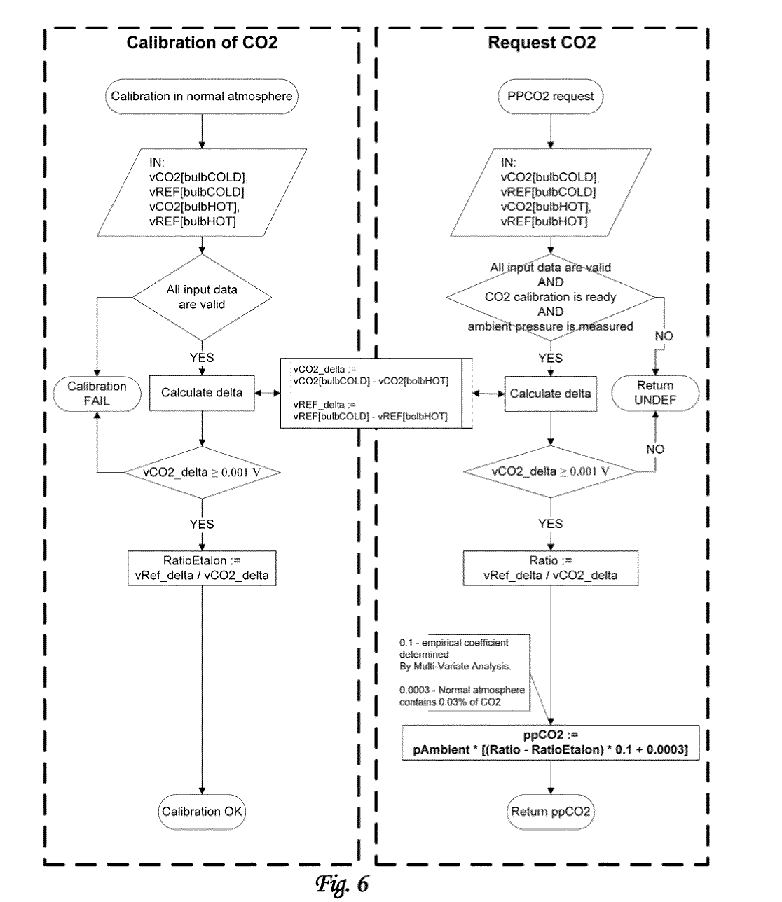

As many gases absorb well in the IR area, it is often necessary to compensate for interfering components by correction block (19). The correction or compensating factors can be obtained by characterising the sensor in the range of environments it may be exposed to, and isolating each of the parameters affecting the accuracy of the result, using normal processes for Multi-Variate Analysis to create a set of Surrogate Models or Response Surface Models that can be applied to the output of the divider (18) to produce a CO2 signal that has an improved signal to noise ratio. The description of a typical calibration and measurement algorithm using UML (Unified Modelling Language) is given in FIG. 6, including a parameter obtained by Multi-Variate Analysis, and the Surrogate Model equation this produced.

It is beneficial to chop or modulate the source generator (12) so that thermal background signals can be offset from the desired signal.

The power consumption of the CO2 sensor assembly generally requires management, by switching the circuit on periodically for a short period to take a measurement, then switching it off. It can be seen from the waveforms in FIG. 3 that there is a minimum time required to obtain the maximum amplitude of signal, typically 360 ms, which means that to take a single on-off measurement will generally require a second. This is not a response time: the response time for the CO2 sensors can be a few tens of milli-seconds using chopper methods or source modulation to remove background thermal drift, but it is a time that the circuit must be on to obtain a measurement with the maximum resolution. The power consumption of a CO2 sensor is difficult to reduce below 170 mA at 3V (510 mW), and if active helium compensation is used in the correction unit (19), this figure can double as helium is measured by measuring the thermal capacity of the gas—such as by heating a resistor and measuring the time it takes to cool by a predetermined percentage. Half to one Watt of power is a large amount of power for a piece of portable equipment.

There are several methods by which the mean power can be reduced. If the sensor is used just once every minute, then this power reduces by a factor of 60, but the effect of a flood in a rebreather, for example, can cause a very rapid escalation of retained CO2 levels which may fall between the sample points from the time it starts to the time where the user suffers a disabling injury. A preferred method of reducing the power consumption is to use a long interval between periodic measurements, but trigger an immediate measurement if there is a change in the user's breathing rate. The breathing rate can be measured easily using a differential sensor in the mouthpiece with a 0.3 Hz low pass filter to remove noise from speech, clicks and fricative breathing noise.

It will be appreciated by a person skilled in the art that the monitor and alarms may be implemented using a micro-controller or gate array, and that various functions within the circuitry would be carried out by that digital logic. Where a rebreather controller contains a suitable micro-controller or gate array, the carbon dioxide monitoring system may be fully integrated within the rebreather controller or combined with another monitor, such as the partial pressure of oxygen (PPO2) monitor that is normally fitted to such equipment.

I claim:

1. A rebreather safety monitoring device comprising:
a carbon dioxide sensor provided with a gas sampler, wherein the gas sampler is adapted for sampling a gas in a rebreather breathing loop from a location between an inhale one-way valve on a rebreather mouthpiece and a carbon dioxide scrubber and providing an obtained gas sample to the carbon dioxide sensor; and
an interface to one selected from the group consisting of an alarm and a warning, wherein the interface is based on a carbon dioxide reading.

2. A monitor according to claim 1, wherein the carbon dioxide sensor is calibrated using a carbon dioxide level in human expired gas.

3. A monitor according to claim 1, wherein the carbon dioxide sensor is fitted with a hydrophobic membrane.

4. A monitor according to claim 1, wherein the carbon dioxide sensor comprises a dual channel infra-red absorption sensor.

5. A monitor according to claim 1 wherein the carbon dioxide sensor is powered up with a low periodic duty cycle, but where an alarm system is powered even when the carbon dioxide sensor is powered down.

6. A monitor according to claim 1 wherein the carbon dioxide reading is powered up and a reading taken when a change in a user's respiratory rate is detected.

7. A monitor according to claim 1 further comprising a pressure sensor that is applied to compensate the carbon dioxide reading for changes in ambient pressure.

8. A monitor according to claim 1 further comprising a helium sensor that is applied to compensate the carbon dioxide reading for changes in the partial pressure of helium.

9. A monitor according to claim 1, wherein the carbon dioxide sensor is a dual channel infra-red absorption sensor with a circuit to drive an infra-red light source with greater power when under ambient pressure or in a presence of gases having a high thermal capacity, than in air at one atmosphere pressure, thus stabilising a spectrum of an emitted light under a range of pressures or in the presence of helium.

10. A monitor according to claim 1, wherein the carbon dioxide sensor comprises a dual channel infra-red absorption sensor with an infra-red emitter isolated from the effects of pressure and helium such that the emitted spectrum does not change by more than 50% under a range of operating pressures or helium the monitor covers.

11. A monitor according to claim 1, wherein an alarm level is used to drive shut a valve that closes the breathing loop.

12. A monitor according to claim 1, wherein an alarm level is used to drive shut a valve that closes the breathing loop at the mouthpiece and switches a user to an alternative gas source.

13. A monitor according to claim 1, wherein an absence of carbon dioxide, or a presence of carbon dioxide at a partial pressure lower than that normally expired by a human, is applied to trigger one selected from the group consisting of a warning or an alarm level.

14. A monitor according to claim 1, wherein a presence of carbon dioxide at a partial pressure higher than that normally expired by a human is applied to trigger one selected from the group consisting of a warning or an alarm level.

15. A monitor according to claim 1, wherein the device is integrated with one selected from the group consisting of a partial pressure of oxygen monitor, a measurement device, and a controller.

16. A monitor according to claim 1, wherein the carbon dioxide sensor is coupled thermally to a scrubber such that it operates at more than 3 degrees Celsius above an ambient temperature.

17. A monitor according to claim 1, that is calibrated automatically when a scrubber canister is opened and a scrubber removed, as indicated by a the presence of light on a light sensor, with a human interface that allows a background level of carbon dioxide to be indicated to a calibration system.
